(19) United States
(12) Patent Application Publication
(10) Pub. No.: US 2022/0118128 A1
(43) Pub. Date: Apr. 21, 2022

(54) ULTRAVIOLET THERAPY WITH WHITE TUNING

(71) Applicant: Lumileds LLC, San Jose, CA (US)

(72) Inventors: Yifeng Qiu, San Jose, CA (US); Noman Rangwala, San Francisco, CA (US)

(21) Appl. No.: 17/502,528

(22) Filed: Oct. 15, 2021

Related U.S. Application Data
(60) Provisional application No. 63/093,950, filed on Oct. 20, 2020.

(51) Int. Cl. A61L 2/08; A61L 2/10; H05B 45/30; H05B 45/28; H05B 47/105; A61L 2/24

(52) U.S. Cl. CPC A61L 2/084; A61L 2/10; H05B 45/30; A61L 2202/14; H05B 47/105; A61L 2/24; A61L 2202/11; H05B 45/28

(57) ABSTRACT

A light-emitting apparatus can provide disinfecting, temperature-variable white light while compensating for a color shift provided by UV-A light. A lighting apparatus can include a plurality of blue light emitting diodes (LEDs), a plurality of red LEDs, a plurality of green LEDs, a plurality of ultraviolet A (UV-A) LEDs, driver circuitry coupled to the plurality of blue LEDs, the plurality of red LEDs, the plurality of green LEDs, and the plurality of UV-A LEDs, and controller circuitry configured to receive a white color temperature and provide signals to the driver circuitry that cause the driver circuitry to drive the plurality of blue LEDs, plurality of red LEDs, plurality of green LEDs, and plurality of UV-A LEDs to generate white light at the white color temperature and compensate for a color shift provided by the plurality of UV-A LEDs.

### CLAIM OF PRIORITY

This application claims the benefit of priority of U.S. Provisional Patent Application No. 63/093,950, filed on Oct. 20, 2020, which is hereby incorporated by reference in its entirety.

### TECHNICAL FIELD

The present disclosure relates to a light-emitting apparatus and a light-emitting apparatus control system configured to provide white point tuning and disinfection capabilities. The light-emitting apparatus can include ultraviolet (UV) light emitting diodes (LEDs), along with color LEDs. The control system for the LEDs adjusts radiometric power and duty cycle of the UV and color LEDs to achieve a desired white color point.

### BACKGROUND

In some applications, such as home or commercial lighting, user experience is very important. Further, color tuning is an integral part of human-centric lighting. Some control technologies offer lighting specifiers that allow end-users new possibilities in lighting control. With an increased awareness of bacteria and viruses, it is desired to have a disinfecting light that includes white light temperature control.

### BRIEF DESCRIPTION OF THE DRAWINGS

The figures show various views of an apparatus, system, or method, including a control system that can alter light emerging from one or more LEDs, in accordance with some embodiments. The terms “front,” “rear,” “top,” “side,” and other directional terms are used merely for convenience in describing the apparatuses and systems and other elements and should not be construed as limiting in any way.

FIG. 1 illustrates, by way of example, a diagram of an embodiment of a system for providing white point tunable disinfecting light.

FIG. 2 illustrates, by way of example, a diagram of an embodiment of another system for providing white point tunable disinfecting light.

FIG. 3 illustrates, by way of example, a diagram of an embodiment of another system for providing white point tunable disinfecting light.

FIGS. 4, 5, 6, and 7 illustrate, by way of example, spectrum content of white light generated at a variety of respective temperatures using an embodiment.

FIG. 8 illustrates, by way of example, a diagram of an embodiment of a method for generating temperature-variable, disinfecting white light that compensates for a color shift from UV-A light.

FIG. 9 illustrates, by way of example, a block diagram of an embodiment of a machine 900 (e.g., a computer system) to implement one or more embodiments.

Corresponding reference characters generally indicate corresponding parts throughout the several views. Elements in the drawings are not necessarily drawn to scale. The configurations shown in the drawings are merely examples and should not be construed as limiting the scope of the disclosed subject matter in any manner.

### DETAILED DESCRIPTION

In addition to correlated color temperature (CCT) tuning over a wide range, the user can change the tint of the white, or other color, light over a CCT area as they desire.

The home and commercial lighting systems, with their wide tuning range on a single platform, are ideal candidates for all kinds of color-tunable applications. A wavelength of UVA light (generally about 315 nm to about 430 nm, but considered to be at about 405 nm for various embodiments described herein) is effective for disinfection under specific dosage conditions. To enable disinfection, the UVA source must be on. However, adding UVA light to existing white sources results in shifts of color point for the white source. Although the 405 nm wavelength light source mostly emits radiation in the non-visible spectrum, the UVA source has a small visible component. Thus, the UVA source does emit partially in the visible spectrum. The UVA source thus affects the color point of light provided alongside with the UVA source.

Table 1 shows the effect of UVA 405 nm LEDs when used together with white emitting LEDs.

TABLE 1
Color Temperature Shift When Including
UVA Source With Color Tunable LEDs

| WHITE LED WITHOUT UV | | WHITE LED WITH UVA ON | |
|---|---|---|---|
| Color | Deep UV (DUV) | Color | DUV |
| Temperature | Content | Temperature | Content |
| 2700 K | 0 | 3150 K | −0.034 |
| 4000 K | 0.001 | 5011 K | −0.021 |
| 6500 K | 0.003 | 9824 K | −0.012 |

For Table 1, in all cases, there are six (6) white LEDs and 6 UVA LEDs. All of the LEDs conduct 0.5 Amps (A) when powered on. Table 1 shows that the shift in color point can be pretty large, with the shift being greater with higher color temperatures. The size of the shift gets bigger as the color temperature gets higher. For example, the shift is 450K when UVA LEDs are powered on along with LEDs that provide color at a temperature of 2700K and the shift is 3324K when UVA LEDs are powered on along with LEDs that provide color at a temperature of 6500K. Not only is the color shift bigger, but it is a bigger proportion of the temperature of the color. Providing a control mechanism to provide a color at an expected color temperature while still providing UVA disinfection is difficult and is what embodiments of the disclosed subject matter provide.

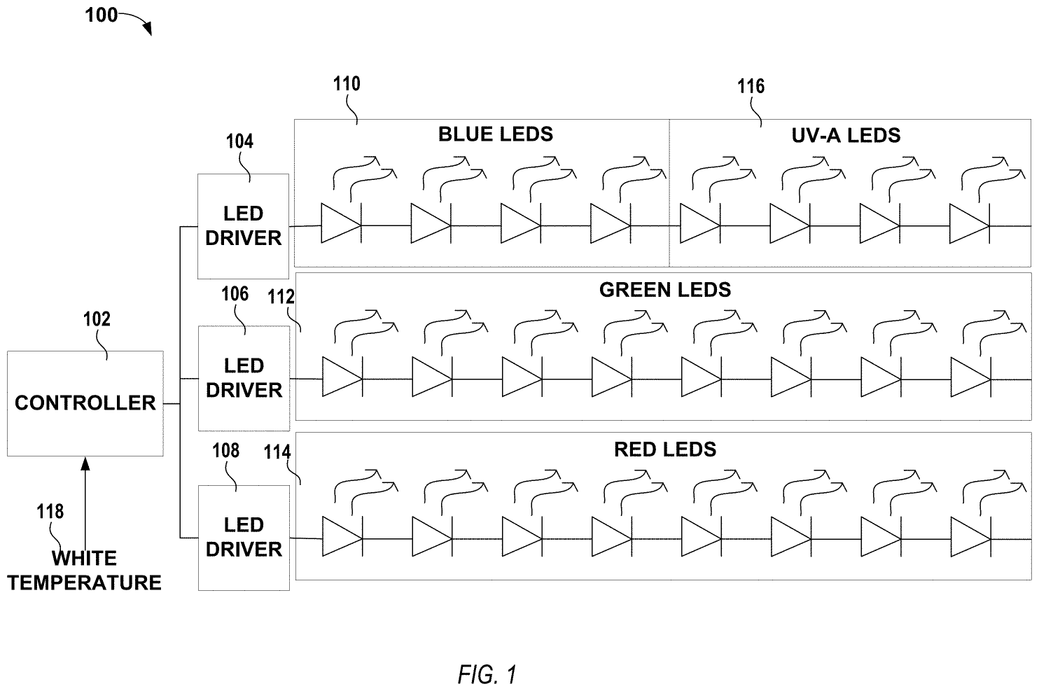

FIG. 1 illustrates, by way of example, a diagram of an embodiment of a system 100 for providing white point tunable disinfecting light. The system 100 as illustrated includes controller circuitry 102, and LED driver circuitry 104, 106, 108 for respective strings of LEDs 112, 114, and 110 and 116. In the embodiment of FIG. 1, some of the blue LEDs 110 are replaced with UV-A LEDs 116. That is, there are fewer blue LEDs 110 than green LEDs 112 or red LEDs 114, but there are still the same number of LEDs in each of the strings of LEDs. FIG. 1 is merely an example and more or fewer LEDs can be used in a string of LEDs.

The LED controller circuitry 102 issues commands to the LED driver circuitry 104, 106, 108 that cause the driver circuitry 104, 106, 108 to operate the corresponding LED strings, a first string including the blue LEDs 110 and UV-A LEDs 116, a second string including the green LEDs 112, and a third string including the red LEDs 114. In general, each LED driver circuitry 104, 106, 108 can operate one string of LEDs.

Circuitry, such as the controller circuitry 102, LED driver circuitry 104, 106, or 108, can include electric or electronic components configured to perform operations of the circuitry. Electric or electronic components can include one or more transistors, resistors, inductors, capacitors, diodes, switches, multiplexers, oscillators, current, voltage, or power supplies, buck or boost converters, modulators, demodulators, analog to digital converters, digital to analog converters, logic gates (e.g., AND, OR, XOR, negate, buffer, or the like), amplifiers, or the like.

The controller circuitry 102 can receive a white temperature 118. The white temperature 118 can be provided by a user of the lighting apparatus, such as through an input device (e.g., a touch screen, slider, potentiometer, button, toggle, a microphone, or the like). The controller circuitry 102 can determine a duty cycle for each of the strings of LEDs that meets the white temperature 118. As previously discussed, if the controller circuitry 102 would use a duty cycle for a normal three-color white light generation scheme to drive the LED strings of FIG. 1, the light would be shifted in temperature (see Table 1). The controller circuitry 102 can compensate the duty cycles of the LED strings such that the white temperature 118 is achieved and the white shift realized by using the UV-A LEDs 116 is compensated for. Table 2 illustrates an example of duty cycle ratios to achieve a few selected white temperatures.

TABLE 2
duty cycle ratios for three color string with UV-A LEDs
replacing some blue LEDs. For the example of Table
2, there are three strings of 12 LEDs, a first string
with 12 red LEDs, a second string with 12 green LEDs,
and a third string with 6 blue LEDs and 6 UV-A LEDs.

| TEMPERATURE (K) | RATIO RED | RATIO GREEN | RATIO BLUE |
|---|---|---|---|
| 2700 | 47% | 45% | 8% |
| 3000 | 39% | 47% | 14% |
| 3500 | 28% | 49% | 23% |
| 4000 | 22% | 47% | 31% |
| 5000 | 13% | 42% | 45% |
| 5700 | 9% | 39% | 53% |
| 6500 | 6% | 35% | 59% |
| 7500 | 5% | 30% | 65% |
| 10000 | 3% | 23% | 74% |

The blue LEDs 110 have a greater photometric lumen compared to UV-A. This lumen difference allows for compensation in the visible UV color range and allowing the driver circuitry 104, 106, 108 to jointly drive the LEDs 110, 112, 114, 116 to provide white light with the specified white temperature 118. Driving the LEDs 110, 112, 114, 116, by the driver circuitry 104, 106, 108 can include driving the LEDs (i) 110 and 116, (ii) 112, and (iii) 114 in sequence, such that only a single string of LEDs is driven at a given time, driving two of (i), (ii), and (iii) in sequence, such that two strings of LEDs are driven at a given time, or driving the LEDs (i), (ii), and (iii) at different electrical powers simultaneously to generate the white light, or a combination thereof. A combination can include using the driving scheme that is more energy efficient if the driving scheme can generate the white temperature 118 and using a different driving scheme otherwise.

The LEDs 110, 112, 114, 116 (or other LEDs discussed herein) can be phosphor converted LEDs, such as can include a phosphor material on a die of the LEDs. The phosphor shift can provide a blue, yellow, green, or red shift in color. The phosphor converted LEDs can have a same forward voltage as LEDs without the phosphor material over the LED die. A phosphor-converted red LED is less temperature dependent than other red LEDs. White light generated by phosphor-converted LEDs is sometimes called phosphor-converted white light.

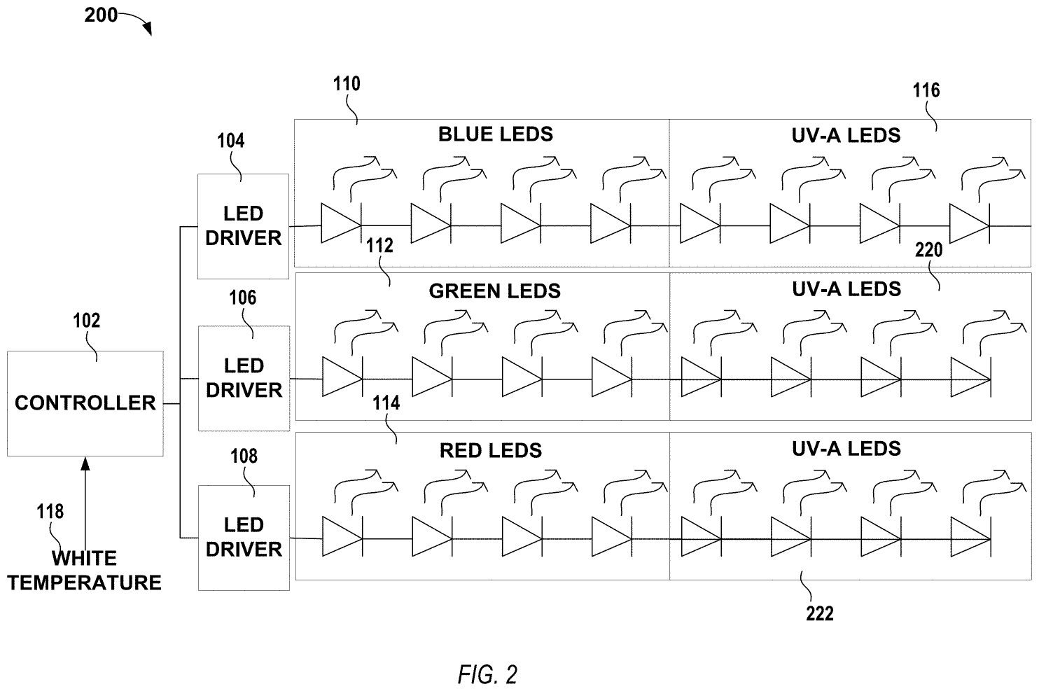

FIG. 2 illustrates, by way of example, a diagram of an embodiment of another system 200 for providing white point tunable disinfecting light. The system 100 as illustrated includes controller circuitry 102, and LED driver circuitry 104, 106, 108 for respective strings of LEDs 110 and 116, 112 and 220, 114 and 222. The controller circuitry 102, and LED driver circuitry 104, 106, 108 may be the same as or similar to the controller circuitry 102, and LED driver circuitry 104, 106, 108 of FIG. 1. In the embodiment of FIG. 2, some of the blue LEDs 110, some of the green LEDs 112, and some of the red LEDs 114 are replaced with UV-A LEDs 116, 220, 222, respectively. That is, there are fewer blue LEDs 110, green LEDs 112, and red LEDs 114 than there are LEDs in each string. Similar to FIG. 1, the system 200 includes the same number of LEDs in each of the strings of LEDs. FIG. 2 is merely an example and more or fewer LEDs can be used in a string of LEDs.

The controller circuitry 102 can receive a white temperature 118 information, such as from a user of the system 200 through an input device (e.g., a touch screen, slider, potentiometer, button, toggle, a microphone, or the like). The controller circuitry 102 can determine a duty cycle for each of the strings of LEDs (a) a first string of LEDs 110 and 116, (b) a second string of LEDs 112 and 220, and (c) a third string of LEDs 114 and 222, that generates the perceived white temperature 118. As previously discussed, if the controller circuitry 102 would use a duty cycle for a normal three color white light generation scheme to drive the LED strings (a), (b), and (c) of FIG. 2, the light would be shifted in temperature in a manner similar to that discussed with regard to FIG. 1. The controller circuitry 102 can compensate the duty cycles of the LED strings (a), (b), and (c) such that the white temperature 118 is achieved and the white shift realized by using the UV-A LEDs 116 is compensated for. Table 3 illustrates an example of duty cycle ratios to achieve a few selected white temperatures using UV-A LEDs in each string (a), (b), and (c) of LEDs. Compared with the system 100 of FIG. 1, the system 200 of FIG. 2 can generate higher doses of UV-A light, such as to provide better disinfection capability.

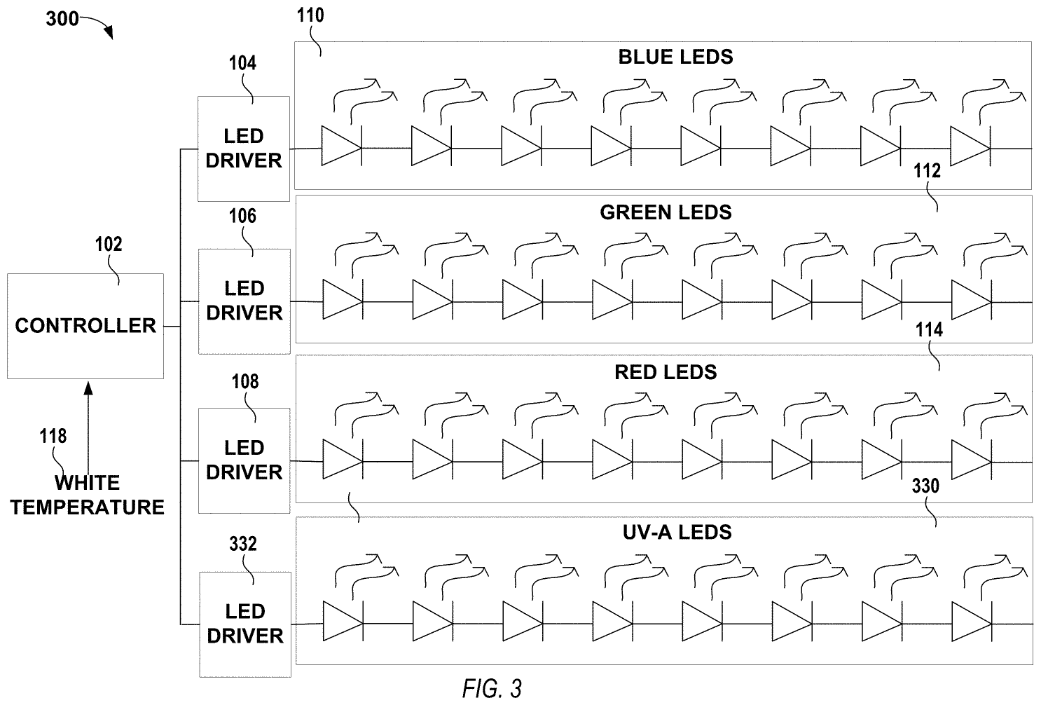

FIG. 3 illustrates, by way of example, a diagram of an embodiment of another system 300 for providing white point tunable disinfecting light. The system 300 as illustrated includes controller circuitry 102, and LED driver circuitry 104, 106, 108, 332 for respective strings of LEDs 110, 112, 114, and 330. The controller circuitry 102, and LED driver circuitry 104, 106, 108 may be the same as or similar to the controller circuitry 102, and LED driver circuitry 104, 106, 108 of FIG. 1. In the embodiment of FIG. 3 the UV-A LEDs 330 are on a dedicated string. That is, there are the same number of green, red, blue, and UV-A LEDs. FIG. 3 is merely an example and more or fewer LEDs can be used in a string of LEDs.

The LED controller circuitry 102 issues commands to the LED driver circuitry 104, 106, 108, 332 that cause the driver circuitry 104, 106, 108, 332 to operate the corresponding LED strings (I) the blue LEDs 110, (II) the green LEDs 112, (III) the red LEDs 114, and (IV) the UV-A LEDs 330. In general, each LED driver circuitry 104, 106, 108, 332 can operate one string of LEDs.

The controller circuitry 102 can receive a white temperature 118. The controller circuitry 102 can receive a white temperature 118 information, such as from a user of the system 200 through an input device (e.g., a touch screen, slider, potentiometer, button, toggle, a microphone, or the like). The controller circuitry 102 can determine a duty cycle for each of the strings of LEDs that meets the white temperature 118. As previously discussed, if the controller circuitry 102 would use a duty cycle for a normal three-color white light generation scheme to drive the LED strings of FIG. 3, the light would be shifted in temperature similar to the systems 100, 200. The controller circuitry 102 can compensate the duty cycles of the LED strings (I), (II), (III) such that the white temperature 118 is achieved and the white shift realized by using the UV-A LEDs 330 is compensated for. Table 4 illustrates an example of duty cycle ratios to achieve a few selected white temperatures using the system 300.

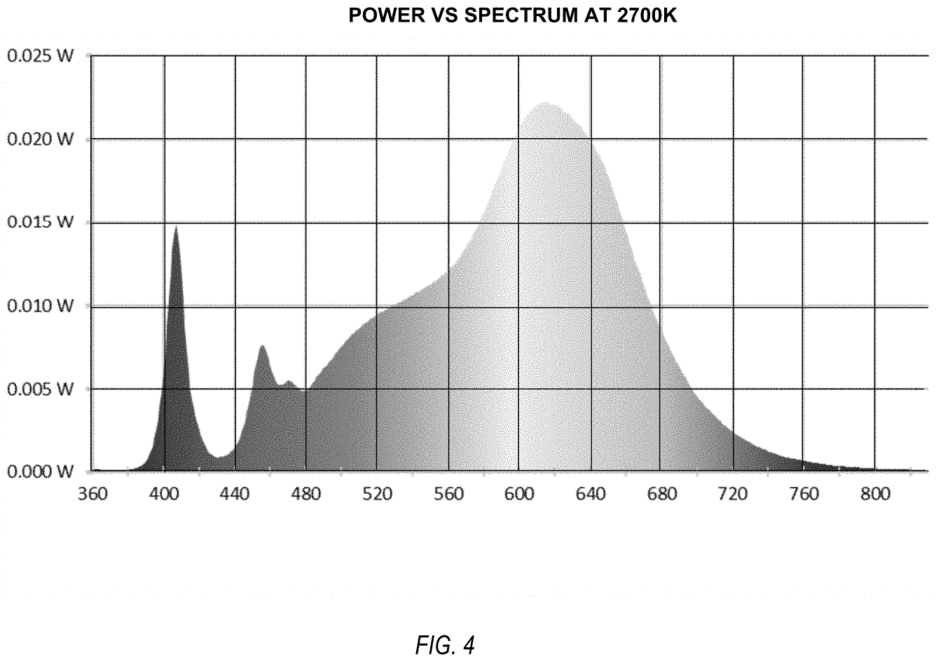

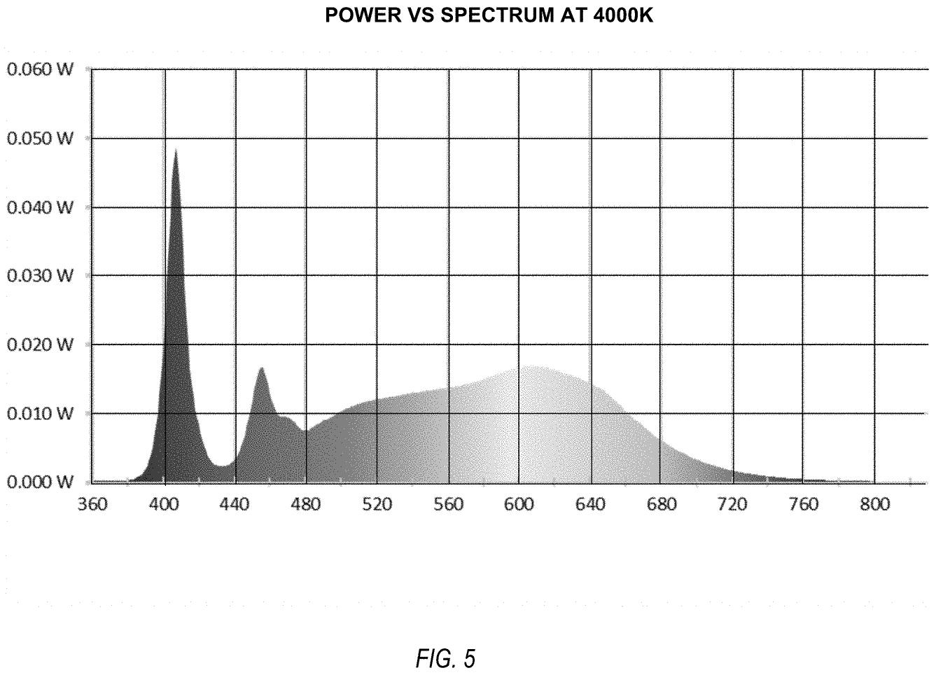

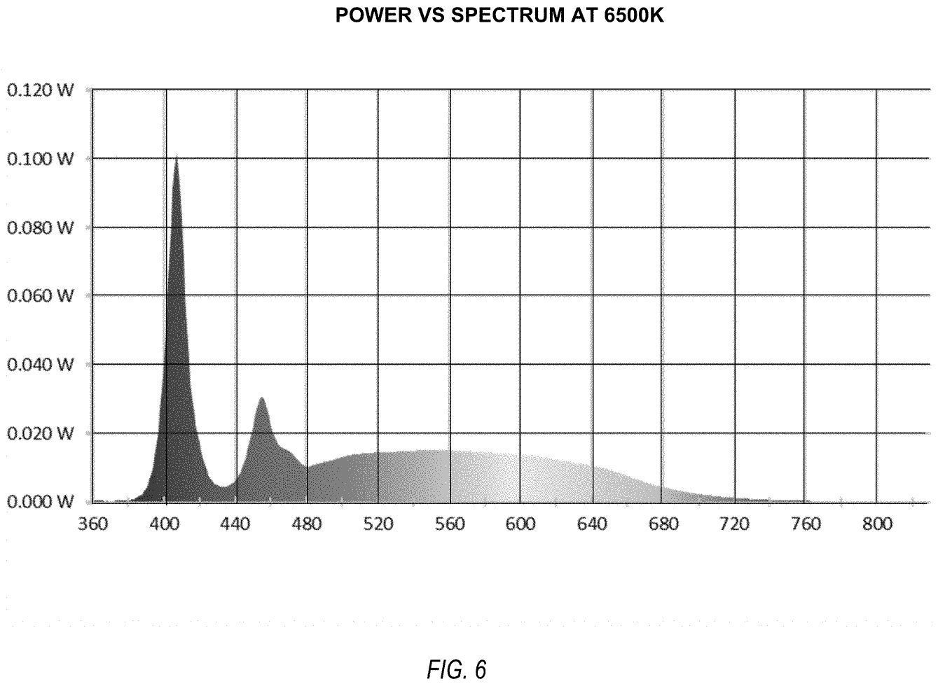

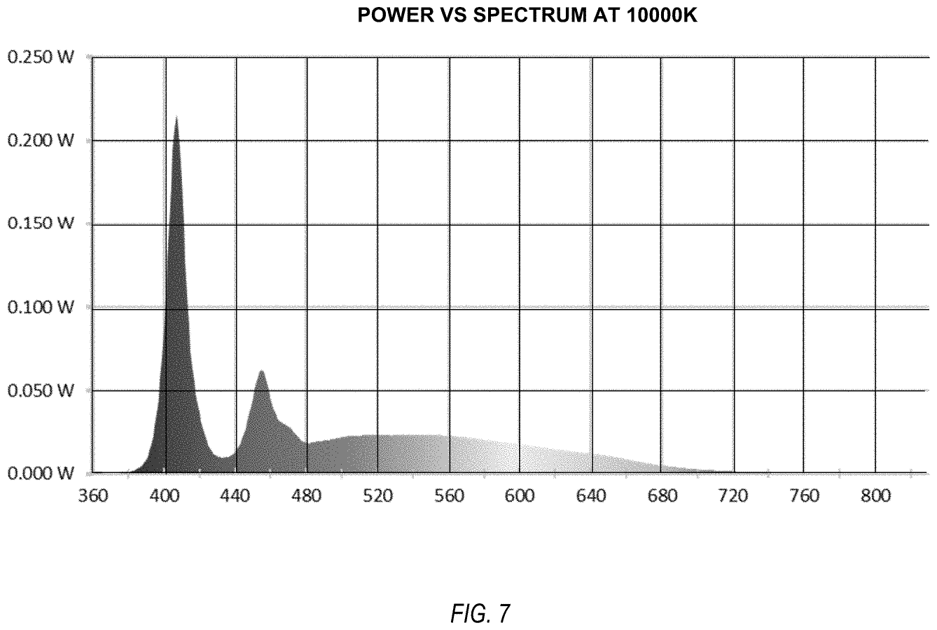

FIGS. 4, 5, 6, and 7 illustrate, by way of example, spectrum content of white light generated at a variety of respective temperatures using an embodiment. FIG. 4 illustrates spectrum content of disinfecting white light at a temperature of 2700K. FIG. 5 illustrates spectrum content of disinfecting white light at a temperature of 4000K. FIG. 6 illustrates spectrum content of disinfecting white light at a temperature of 6500K. FIG. 7 illustrates spectrum content of disinfecting white light at a temperature of 10000K.

Comparing FIG. 4-7 one can realize that the higher the temperature, the more powerful the UV-A content of the light. Thus, the warmth of the light can indicate the disinfecting properties of the white light.

Note that the ability to compensate for UV-A content in the white light depends on the power of UV-A light. If the UV-A is provided on a blue LED string, the power of the UV-A is generally lower than UV-A provided on a green or red LED string. This is because the green and red LED strings are typically driven at higher power. To compensate for this effect, fewer UV-A LEDs can be included in a red or green LED string than would be included in the blue LED string. The UV-A LEDs can be included in any combination of the blue, red, and green LED strings as long as the total power of the UV-A can be compensated for by adjusting the duty cycles of the red, green, and blue LED strings accordingly to achieve the same white color point.

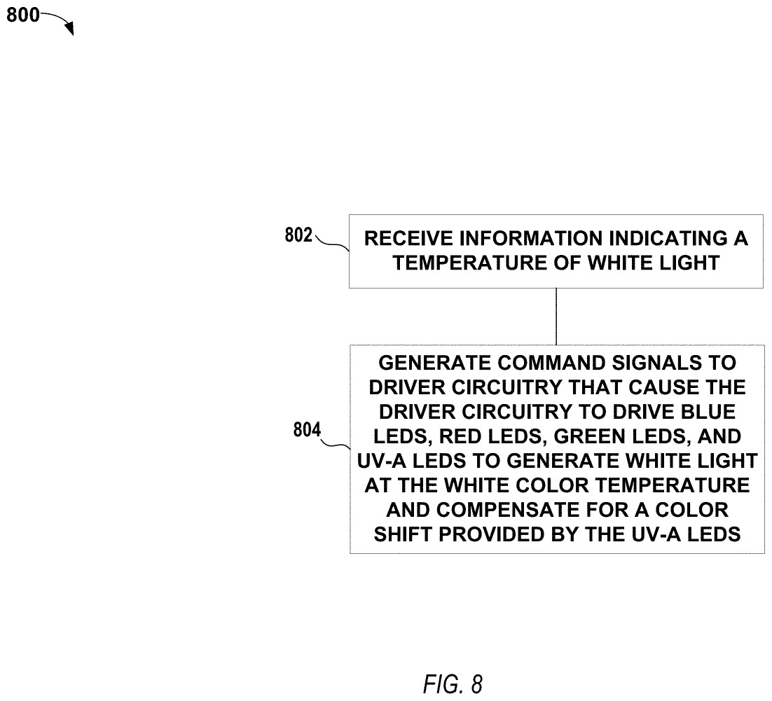

FIG. 8 illustrates, by way of example, a diagram of an embodiment of a method 800 for generating temperature-variable, disinfecting white light that compensates for a color shift from UV-A light. The method 800 can be performed by LED control circuitry 102. The method 800 as illustrated includes receiving, at controller circuitry, information indicating a temperature of white light, at operation 802; and generating command signals to driver circuitry that cause the driver circuitry to drive a plurality of blue light emitting diodes (LEDs), a plurality of red LEDs, a plurality of green LEDs, and a plurality of UV-A LEDs to generate white light at the white color temperature and compensate for a color shift provided by the plurality of UV-A LEDs, at operation 804.

The method 800 can further include, wherein the plurality of blue LEDs, plurality of red LEDs, and plurality of green LEDs are phosphor-converted LEDs. The method 800 can further include, wherein the driver circuitry includes first driver circuitry, second driver circuitry, and third driver circuitry dedicated to driving first, second, and third LED strings, respectively. The method 800 can further include, wherein the plurality of blue LEDs and plurality of UV-A LEDs are on the first LED string, the plurality of green LEDs are on the second LED string, and the plurality of red LEDs are on the third LED string. The method 800 can further include, wherein the plurality of blue LEDs and a first subset of the plurality of UV-A LEDs are the first LED string, the plurality of green LEDs and a second subset of the plurality of UV-A LEDs are the second LED string, and the plurality of red LEDs and a third subset of the plurality of UV-A LEDs are the third LED string.

The method 800 can further include, wherein the driver circuitry further includes fourth driver circuitry dedicated to driving a fourth LED string. The method 800 can further include, wherein the plurality of blue LEDs are on the first LED string, the plurality of green LEDs are on the second LED string, the plurality of red LEDs are on the third LED string, and the plurality of UV-A LEDs are on the fourth LED string.

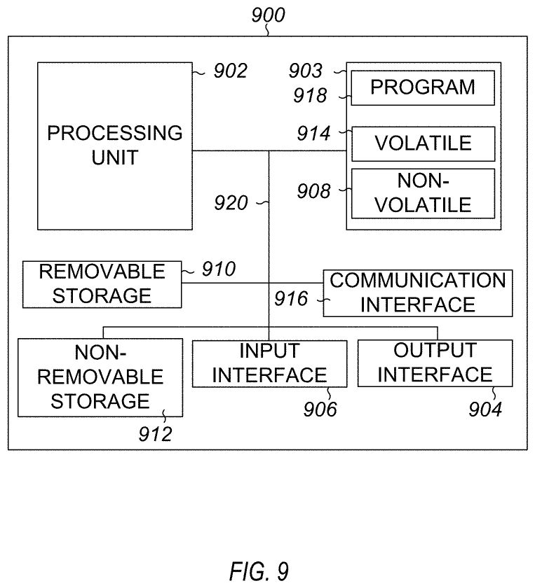

FIG. 9 illustrates, by way of example, a block diagram of an embodiment of a machine 900 (e.g., a computer system) to implement one or more embodiments. One or more of the controller circuitry 102 or driver circuitry 104, 106, 108, 332 can include one or more of the components of the machine 900. One or more of the controller circuitry 102 or driver circuitry 104, 106, 108, 332 can be coupled to one or more of the components of the machine 900. One example machine 900 (in the form of a computer), may include a processing unit 902, memory 903, removable storage 910, and non-removable storage 912. Although the example computing device is illustrated and described as machine 900, the computing device may be in different forms in different embodiments. For example, the computing device may instead be a smartphone, a tablet, smartwatch, or other computing device including the same or similar elements as illustrated and described regarding FIG. 9. Devices such as smartphones, tablets, and smartwatches are generally collectively referred to as mobile devices. Further, although the various data storage elements are illustrated as part of the machine 900, the storage may also or alternatively include cloud-based storage accessible via a network, such as the Internet.

Memory 903 may include volatile memory 914 and non-volatile memory 908. The machine 900 may include—or have access to a computing environment that includes—a variety of computer-readable media, such as volatile memory 914 and non-volatile memory 908, removable storage 910 and non-removable storage 912. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices capable of storing computer-readable instructions for execution to perform functions described herein.

The machine 900 may include or have access to a computing environment that includes input 906, output 904, and a communication connection 916. Output 904 may include a display device, such as a touchscreen, that also may serve as an input device. The input 906 may include one or more of a touchscreen, touchpad, mouse, keyboard, camera, one or more device-specific buttons, one or more sensors integrated within or coupled via wired or wireless data connections to the machine 900, and other input devices. The computer may operate in a networked environment using a communication connection to connect to one or more remote computers, such as database servers, including cloud based servers and storage. The remote computer may include a personal computer (PC), server, router, network PC, a peer device or other common network node, or the like. The communication connection may include a Local Area Network (LAN), a Wide Area Network (WAN), cellular, Institute of Electrical and Electronics Engineers (IEEE) 802.11 (Wi-Fi), Bluetooth, or other networks.

Computer-readable instructions stored on a computer-readable storage device are executable by the processing unit 902 of the machine 900. A hard drive, CD-ROM, and RAM are some examples of articles including a non-transitory computer-readable medium such as a storage device. For example, a computer program 918 may be used to cause processing unit 902 to perform one or more methods or algorithms described herein.

To further illustrate the apparatus and related method disclosed herein, a non-limiting list of examples is provided below. Each of the following non-limiting examples can stand on its own or can be combined in any permutation or combination with any one or more of the other examples.

In Example 1, a disinfecting, temperature-variable white light emitting apparatus comprising a plurality of blue light emitting diodes (LEDs), a plurality of red LEDs, a plurality of green LEDs, a plurality of ultraviolet A (UV-A) LEDs, driver circuitry coupled to the plurality of blue LEDs, the plurality of red LEDs, the plurality of green LEDs, and the plurality of UV-A LEDs, and controller circuitry configured to receive a white color temperature and provide signals to the driver circuitry that cause the driver circuitry to drive the plurality of blue LEDs, plurality of red LEDs, plurality of green LEDs, and plurality of UV-A LEDs to generate white light at the white color temperature and compensate for a color shift provided by the plurality of UV-A LEDs.

In Example 2, Example 1 further includes, wherein the plurality of blue LEDs, plurality of red LEDs, and plurality of green LEDs are phosphor-converted LEDs.

In Example 3, at least one of Examples 1-2 further includes, wherein the driver circuitry includes first driver circuitry, second driver circuitry, and third driver circuitry dedicated to driving first, second, and third LED strings, respectively.

In Example 4, Example 3 further includes, wherein the plurality of blue LEDs and plurality of UV-A LEDs are on the first LED string, the plurality of green LEDs are on the second LED string, and the plurality of red LEDs are on the third LED string.

In Example 5, Example 3 further includes, wherein the plurality of blue LEDs and a first subset of the plurality of UV-A LEDs are the first LED string, the plurality of green LEDs and a second subset of the plurality of UV-A LEDs are the second LED string, and the plurality of red LEDs and a third subset of the plurality of UV-A LEDs are the third LED string.

In Example 6, Example 3 further includes, wherein the driver circuitry further includes fourth driver circuitry dedicated to driving a fourth LED string.

In Example 7, Example 6 further includes, wherein the plurality of blue LEDs are on the first LED string, the plurality of green LEDs are on the second LED string, the plurality of red LEDs are on the third LED string, and the plurality of UV-A LEDs are on the fourth LED string.

Example 8 includes a method of generating disinfecting, temperature-variable white light, the method comprising receiving, at controller circuitry, information indicating a temperature of white light, and generating, by the controller circuitry, command signals to driver circuitry that cause the driver circuitry to drive a plurality of blue light emitting diodes (LEDs), a plurality of red LEDs, a plurality of green LEDs, and a plurality of UV-A LEDs to generate white light at the white color temperature and compensate for a color shift provided by the plurality of UV-A LEDs.

In Example 9, Example 8 can further include, wherein the plurality of blue LEDs, plurality of red LEDs, and plurality of green LEDs are phosphor-converted LEDs.

In Example 10, at least one of Examples 8-9 can further include, wherein the driver circuitry includes first driver circuitry, second driver circuitry, and third driver circuitry dedicated to driving first, second, and third LED strings, respectively.

In Example 11, Example 10 can further include, wherein the plurality of blue LEDs and plurality of UV-A LEDs are on the first LED string, the plurality of green LEDs are on the second LED string, and the plurality of red LEDs are on the third LED string.

In Example 12, Example 10 can further include, wherein the plurality of blue LEDs and a first subset of the plurality of UV-A LEDs are the first LED string, the plurality of green LEDs and a second subset of the plurality of UV-A LEDs are the second LED string, and the plurality of red LEDs and a third subset of the plurality of UV-A LEDs are the third LED string.

In Example 13, Example 10 can further include, wherein the driver circuitry further includes fourth driver circuitry dedicated to driving a fourth LED string.

In Example 14, Example 13 can further include, wherein the plurality of blue LEDs are on the first LED string, the plurality of green LEDs are on the second LED string, the plurality of red LEDs are on the third LED string, and the plurality of UV-A LEDs are on the fourth LED string.

Example 15 includes a tangible machine-readable medium including instructions that, when executed by controller circuitry of a lighting apparatus, cause the lighting apparatus to perform operations for generating disinfecting, temperature-variable white light, the operations comprising receiving information indicating a temperature of white light, and generating command signals to driver circuitry that cause the driver circuitry to drive a plurality of blue light emitting diodes (LEDs), a plurality of red LEDs, a plurality of green LEDs, and a plurality of UV-A LEDs to generate white light at the white color temperature and compensate for a color shift provided by the plurality of UV-A LEDs.

In Example 16, Example 15 can further include, wherein the plurality of blue LEDs, plurality of red LEDs, and plurality of green LEDs are phosphor-converted LEDs.

In Example 17, at least one of Examples 15-16 can further include, wherein the driver circuitry includes first driver circuitry, second driver circuitry, and third driver circuitry dedicated to driving first, second, and third LED strings, respectively.

In Example 18, Example 17 can further include, wherein the plurality of blue LEDs and plurality of UV-A LEDs are on the first LED string, the plurality of green LEDs are on the second LED string, and the plurality of red LEDs are on the third LED string.

In Example 19, Example 17 can further include, wherein the plurality of blue LEDs and a first subset of the plurality of UV-A LEDs are the first LED string, the plurality of green LEDs and a second subset of the plurality of UV-A LEDs are the second LED string, and the plurality of red LEDs and a third subset of the plurality of UV-A LEDs are the third LED string.

In Example 20, Example 17 can further include, wherein the driver circuitry further includes fourth driver circuitry dedicated to driving a fourth LED string.

In Example 21, Example 20 can further include, wherein the plurality of blue LEDs are on the first LED string, the plurality of green LEDs are on the second LED string, the plurality of red LEDs are on the third LED string, and the plurality of UV-A LEDs are on the fourth LED string.

While example embodiments of the present disclosed subject matter have been shown and described herein, it will be obvious to those skilled in the art that such embodiments are provided by way of example only. Numerous variations, changes, and substitutions will now occur to those skilled in the art, upon reading and understanding the material provided herein, without departing from the disclosed subject matter. It should be understood that various alternatives to the embodiments of the disclosed subject matter described herein may be employed in practicing the various embodiments of the subject matter. It is intended that the following claims define the scope of the disclosed subject matter and that methods and structures within the scope of these claims and their equivalents be covered thereby.

What is claimed is:

1. A disinfecting, temperature-variable white light emitting apparatus comprising:
a plurality of blue light emitting diodes (LEDs);
a plurality of red LEDs;
a plurality of green LEDs;
a plurality of ultraviolet A (UV-A) LEDs;
driver circuitry coupled to the plurality of blue LEDs, the plurality of red LEDs, the plurality of green LEDs, and the plurality of UV-A LEDs; and
controller circuitry configured to receive a white color temperature and provide signals to the driver circuitry that cause the driver circuitry to drive the plurality of blue LEDs, plurality of red LEDs, plurality of green LEDs, and plurality of UV-A LEDs to generate white light at the white color temperature and compensate for a color shift provided by the plurality of UV-A LEDs.

2. The apparatus of claim 1, wherein the plurality of blue LEDs, plurality of red LEDs, and plurality of green LEDs are phosphor-converted LEDs.

3. The apparatus of claim 1, wherein the driver circuitry includes first driver circuitry, second driver circuitry, and third driver circuitry dedicated to driving first, second, and third LED strings, respectively.

4. The apparatus of claim 3, wherein the plurality of blue LEDs and plurality of UV-A LEDs are on the first LED string, the plurality of green LEDs are on the second LED string, and the plurality of red LEDs are on the third LED string.

5. The apparatus of claim 3, wherein the plurality of blue LEDs and a first subset of the plurality of UV-A LEDs are the first LED string, the plurality of green LEDs and a second subset of the plurality of UV-A LEDs are the second LED string, and the plurality of red LEDs and a third subset of the plurality of UV-A LEDs are the third LED string.

6. The apparatus of claim 3, wherein the driver circuitry further includes fourth driver circuitry dedicated to driving a fourth LED string.

7. The apparatus of claim 6, wherein the plurality of blue LEDs are on the first LED string, the plurality of green LEDs are on the second LED string, the plurality of red LEDs are on the third LED string, and the plurality of UV-A LEDs are on the fourth LED string.

8. A method of generating disinfecting, temperature-variable white light, the method comprising:
receiving, at controller circuitry, information indicating a temperature of white light; and
generating, by the controller circuitry, command signals to driver circuitry that cause the driver circuitry to drive a plurality of blue light emitting diodes (LEDs), a plurality of red LEDs, a plurality of green LEDs, and a plurality of UV-A LEDs to generate white light at the white color temperature and compensate for a color shift provided by the plurality of UV-A LEDs.

9. The method of claim 8, wherein the plurality of blue LEDs, plurality of red LEDs, and plurality of green LEDs are phosphor-converted LEDs.

10. The method of claim 8, wherein the driver circuitry includes first driver circuitry, second driver circuitry, and third driver circuitry dedicated to driving first, second, and third LED strings, respectively.

11. The method of claim 10, wherein the plurality of blue LEDs and plurality of UV-A LEDs are on the first LED string, the plurality of green LEDs are on the second LED string, and the plurality of red LEDs are on the third LED string.

12. The method of claim 10, wherein the plurality of blue LEDs and a first subset of the plurality of UV-A LEDs are the first LED string, the plurality of green LEDs and a second subset of the plurality of UV-A LEDs are the second LED string, and the plurality of red LEDs and a third subset of the plurality of UV-A LEDs are the third LED string.

13. The method of claim 10, wherein the driver circuitry further includes fourth driver circuitry dedicated to driving a fourth LED string.

14. The method of claim 13, wherein the plurality of blue LEDs are on the first LED string, the plurality of green LEDs are on the second LED string, the plurality of red LEDs are on the third LED string, and the plurality of UV-A LEDs are on the fourth LED string.

15. A tangible machine-readable medium including instructions that, when executed by controller circuitry of a lighting apparatus, cause the lighting apparatus to perform operations for generating disinfecting, temperature-variable white light, the operations comprising:
receiving information indicating a temperature of white light; and
generating command signals to driver circuitry that cause the driver circuitry to drive a plurality of blue light emitting diodes (LEDs), a plurality of red LEDs, a plurality of green LEDs, and a plurality of UV-A LEDs to generate white light at the white color temperature and compensate for a color shift provided by the plurality of UV-A LEDs.

16. The tangible machine-readable medium of claim 15, wherein the plurality of blue LEDs, plurality of red LEDs, and plurality of green LEDs are phosphor-converted LEDs.

17. The tangible machine-readable medium of claim 15, wherein the driver circuitry includes first driver circuitry, second driver circuitry, and third driver circuitry dedicated to driving first, second, and third LED strings, respectively.

18. The tangible machine-readable medium of claim 17, wherein the plurality of blue LEDs and plurality of UV-A LEDs are on the first LED string, the plurality of green LEDs are on the second LED string, and the plurality of red LEDs are on the third LED string.

19. The tangible machine-readable medium of claim 17, wherein the plurality of blue LEDs and a first subset of the plurality of UV-A LEDs are the first LED string, the plurality of green LEDs and a second subset of the plurality of UV-A LEDs are the second LED string, and the plurality of red LEDs and a third subset of the plurality of UV-A LEDs are the third LED string.

20. The tangible machine-readable medium of claim 17, wherein the driver circuitry further includes fourth driver circuitry dedicated to driving a fourth LED string.

21. The tangible machine-readable medium of claim 20, wherein the plurality of blue LEDs are on the first LED string, the plurality of green LEDs are on the second LED string, the plurality of red LEDs are on the third LED string, and the plurality of UV-A LEDs are on the fourth LED string.
